(19) United States
(12) Patent Application Publication
(10) Pub. No.: US 2021/0386093 A1
(43) Pub. Date: Dec. 16, 2021

(54) METHODS OF USE & COMPOSITIONS FOR OBESITY

(71) Applicant: John Malcolm Hall GREGG, Princeton, NJ (US)

(72) Inventor: John Malcolm Hall GREGG, Princeton, NJ (US)

(21) Appl. No.: 16/326,455

(22) PCT Filed: Aug. 20, 2017
(86) PCT No.: PCT/US2017/047705

§ 371 Date: Feb. 19, 2019

(51) Int. Cl. A23K 10/18; A61K 35/741; A61K 45/06; C12Q 1/02; C12Q 1/04

(52) U.S. Cl. CPC A23K 10/18; A61K 35/741; C12Q 1/04; C12Q 1/025; A61K 45/06

(57) ABSTRACT

This invention relates to the development of prebiotic and probiotic products that treat obesity and overweight conditions and allow people with these tendencies to covert food to lean muscle mass rather than fat.

### CROSS-REFERENCE TO RELATED APPLICATIONS

This application is a national phase entry under 35 U.S.C. § 371 of International Application PCT/US2017/047705, filed Aug. 20, 2017, which claims the benefit under 35 U.S.C. § 119(e) of U.S. Provisional Application No. 62/494,764 filed Aug. 20, 2016, all of which are herein incorporated by reference in their entireties.

### BACKGROUND

Obesity is a huge public health problem in the United States and around the world. There are hundreds of millions of obese people around the world and this condition is associated with a multitude of co-morbid health problems including cardiovascular disease, diabetes, and joint problems associated with the stress of carrying excess weight.

In addition to those people carrying excess weight who are defined as obese, there are millions of additional people around the world who are overweight. Many of these people are prediabetic, diabetic, hypertensive, have metabolic syndrome, and have some degree of congestive heart failure or angina. Some also have fatty liver disease and a number of other medical problems associated with excess weight.

Weight gain has historically been associated with the notion that individuals are consuming too much high caloric food in relation to the amount of food needed to provide energy to the body. In this case, the amount of exercise is not sufficient to utilize and consume the energy provided by the food consumed. Fat is a means of storage of fuel that can provide metabolic energy.

It has been thought that individuals have a metabolic rate that is determined by genetics and age, with the idea that metabolism is slowed with age resulting in an age-related increase in weight over time. It is thought that teenagers have the fastest metabolic rate and thus can consume more food without weight gain and often have this need. Despite this, the rates of childhood and teenage obesity and overweight status has been skyrocketing.

Global trends have demonstrated dramatically increasing rates of overweight status and obesity in populations that have not historically had this problem, like Native Americans. This phenomena has been attributed to change in diets from more aboriginal types of diets containing nuts, berries, and roots, and some meat protein to diets containing lots of carbohydrates, sugar, high fat, and red meat animal protein.

In societies like China, the patterns of weight related chronic disease as well as cancer types have changed from the movement from traditional Asian diets to those that are more Western and typical of richer European and North American countries. With the change of diet a parallel increase has occurred in the correlated obesity and medical morbidities.

New evidence has emerged that metabolism is not solely dependent upon an individual's metabolism resulting from their personal genetic endowment, exercise, and dietary choices. It is also related to the commensal bacteria that have a symbiotic relationship with their human hosts in aiding to digest and process consumed food. These bacteria are resident in different gastro-intestinal ecological systems called, “microbiomes.” These microbiomes consist of balances of different species and strains of bacteria, viruses, fungi, protozoa, and sometimes helminths.

Different microbiomes exist in different parts of the human gastrointestinal system including the oral cavity, stomach, small intestines, and colon. They serve as an assembly line for processing ingested food in combination with enzymes and acids produced by human cells. The bacteria benefit by feeding on the ingested food and the human host benefits by the metabolic processing conducted by the bacteria. In this way humans are holobionts that are made up of both their own genomic material and the genomic material of the microbiota that are present in the various microbiomes which together make up the “hologenome.”

The hologenomes of humans are not constant and undergo temporal shift as community assembly changes over time to do invasion, infection, and colonization in the microbiomes of microbiota that the host human is exposed to. Research suggests an age related ecological succession of bacterial composition of the microbiomes that is correlated with changes in metabolism and weight gain.

While food acts as a prebiotic input that will impact the relative abundance of the bacterial species that are present in the epithelial, mucosal, and luminal compartments of the different gastrointestinal microbiomes and probably results in stable balances of microbiota over time with consistent diet, there are other factors that can rapidly change compositional diversity. These are antibiotics, probiotics, infection, and drugs that impact biological compartment barriers or compositional diversity.

There are only several types of species (bifidobacteria and lactobacilli) in marketed probiotics, most of which do not colonize human hosts in a stable manner. Food as a prebiotic input affects compositional diversity and relative abundance relatively gradually. While infection can lead to pathogen overgrowth of bacterial species and acute morbidity and sometimes mortality, a major disrupter of stabile bacterial communities in GI microbiomes are antibiotics.

The extent of long-term impact of GI microbiomes of episodic antibiotic use (by antimicrobial class) through time is controversial due to mechanisms of bacterial resilience and reconstitution of original compositional diversity and relative abundance. However in livestock animal models, it has been demonstrated empirically over a diverse animal species such as ruminant cattle, monogastric swine, and avian poultry systems that a wide range of antibiotics with very different spectrums of antibacterial activity have a growth promotion effect of deposition of lean muscle mass with feed with the chronic administration of low dose or full doses of antibiotics.

The present invention leverages this demonstrated phenomena by a means of identifying the keystone bacterial species that are benefited or disadvantaged by the administration of these antibiotics to have the phenotypic effect of the building of lead muscle with food intake rather than fat.

The identified keystone species of bacteria produce metabolic products that have the effect of deposition of lean muscle rather than fat with food intake. The present inventions encompasses metabolic products that are produced by these keystone bacterial species that can then be produced directly in a bioreactor where they are secreted and engineered synthetically to have optimal biological effect in humans. Combinations of these metabolic products (we call “bolsols”), and sequences of these bolsols that are processed sequentially in the stomach, small intestines, and colon, we call GI metabolic solutions or “Metasols.”

Mechanisms of bacterial resilience, including the germination of previously environmentally released spores that are ingested, as well as planktonic bacteria shed from biofilm reservoirs, restore the original microbiome long after antibiotics have been discontinued. In some cases the resilience will restore a homeostasis that was reached prior to events that caused a dysbiosis with bacterial overgrowth of particular species, or strains of those species. This resilience and restoration of previous equilibrium can also arrest continuing phenotypic effects from antibiotic administration, including weight gain by the deposition of lean muscle mass that has been observed with continuous administration of regular dose, or low dose, antibiotics over time.

While at least 13 different classes of antibiotics, all with different spectrums of antibacterial activity, have been used in the main types of food production species (cattle, swine, poultry, and fish), to promote production efficiency, none of their enterotypes associated with specific feed regimens have been systematically studied and characterized, and thus the metabolic solutions (combinations of metabolic products of key microbiota species and strains) have not been previously described in detail following metabolomic analysis, nor have metabolic products in the form of probiotics (live bacterial therapeutics) or prebiotics, been developed to produce the same phenotypic effects of the antibiotic modulated enterotypes.

Bioinformatic analysis across three major types of livestock production species, avian (poultry), ruminant (cattle), and monogastric (swine), for the 13 classes of antibiotics used to achieve lean muscle weight gain phenotypes, will provide enough intersection of species variables in the big data of compositional diversity analysis to give a clear signal of keystone species identification for desired processing of food to produce lean muscle rather than fat. Simultaneous equations can be solved to break the metabolic code of which genes are important in the keystone bacterial species and the metabolic products encoded by those genes.

### SUMMARY OF THE INVENTION

The present invention is a set of compositions for more producing lean muscle mass in humans, rather than fat, to mitigate obesity and fat related weight gain and the processes and methods of developing these compositions.

The present invention includes compositions that are administered enterally and by drug or supplement that are metabolic solutions (hereby defined as “Bolsols”) for digesting different foods and optimizing their energy conversion for people and its deposition of lean muscle mass rather than fat. These compositions are artificially derived subsets and mixtures of metabolic products that come from larger metabolic solution recipes encoded in the genomes of unique sets of microbiota combined with genomes of the human hosts (known as the “hologenome of the holobiome”).

The compositions in the present invention are made up components that include, but are not limited to, particular vitamins (e.g., K₁), proteins e.g., trypsin), fatty acids (including short chain fatty acids like butyrate), and sugars (e.g., glucose), in specific set of combinations, as well as compounds screened to modulate microbiota in various gastrointestinal microbiomes (e.g. those found in the stomachs, small intestines, & colons) to produce these component combinations with the desired phenotypic effect of deposition of lean muscle mass.

In another specific embodiment, the present invention includes compounds and/or drugs that have been screened to recapitulate the target enterotype that exhibits the desired phenotypic effect of deposition of lean muscle rather than fat.

### DETAILED DESCRIPTION OF THE INVENTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

Where appropriate, any one or more of the other active agents may be in the form of a pharmaceutically acceptable salt.

Suitable pharmaceutically acceptable salts include, but are not limited to, salts of pharmaceutically acceptable inorganic acids such as hydrochloric, sulfuric, phosphoric, nitric, carbonic, boric, sulfuric, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

It is intended that the aspects and embodiments of this invention encompasses all solid forms, including amorphous forms, as well as crystalline forms, and polymorphs thereof.

Throughout this specification the term ‘in combination’ means that one or more other actives are both administered to people over the same period of treatment. They may be administered together, i.e. at the same time. In this case they may be administered in a single formulation, (e.g. as a single tablet or capsule) or in separate formulations administered simultaneously or nearly simultaneously. Alternatively, they may be administered at separate times of day.

The combinations of the invention provide benefits which are at least additive compared to the use of either agent alone. In many embodiments, the combinations are something more than additive, e.g. synergistic, compared to the use of either agent alone.

The definition of the term ‘treatment’ in this specification encompasses deposition of lean muscle mass rather than fat as well as this purpose plus disease treatment, prophylaxis and prevention (i.e. reducing or eliminating the risk of contracting the disease). As well as meaning curing people of the disease, ‘treatment’ also includes preventing the onset of symptoms, controlling (e.g. by slowing or eliminating) progression of disease, preventing the spread of the disease to other parts of the body and/or to other persons, reducing the spread of the disease and other facets of medical practice which will be readily understood by the person skilled in the art to fall within the meaning of the term ‘treatment’.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

### Formulations

For the above-mentioned therapeutic uses, the dosage administered will, of course, vary with the compound employed, the mode of administration, the treatment desired and the purpose or disorder indicated.

Compositions may be administered systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

For oral administration, one or more active agents may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, one or more active agents may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

### Methods of Bioinformatics

The bioinformatics of the present invention employs for metagenomic microbiota analysis a base of both 16S ribosomal RNA fingerprinting as well as whole genome shotgun sequencing (WGS) analysis of metagenomic data. The WGS in the present invention includes techniques that use iterative scanning of small motifs, including 12 amino-acid (36 bp) motifs, that are then compared for a comprehensive taxonomy against all 280,000 named organisms in public databases and are benchmarked against other pipelines (e.g., MetaPhlan, Phylosift, GOTTCHA and Kraken).

The present invention includes cross-species metagenomic, proteomic, transcriptomic, and metabolomic analyses of the various GI microbiomes (e.g., stomach, small intestines, colon) for each livestock production species, including, but not limited to cattle, poultry (including raised game birds), swine, fish (including farmed fish), venison, bison, sheep, and goats, both before and after administration of an antibiotic, including, but not limited to, those in the class of aminoglycosides, cephalosporins, cyclic peptides, diterpines, fluoroquinolones, hydrazines, ionophores, lincosamides, macrolides, organoarsenics, nitroimidazoles, penicillins, streptogramins, and sulfonamides in combination with various feeds used for those production species.

The present invention also includes cross-species metagenomic, proteomic, transcriptomic, and metabolomic analyses of the various GI microbiomes (e.g., stomach, rumen, small intestines, colon) for each livestock production species, including, but not limited to cattle, poultry (including raised game birds), swine, fish (including farmed fish), venison, bison, sheep, and goats, both before and after administration of combinations of antibiotics, including, but not limited to, those in the class of aminoglycosides, cephalosporins, cyclic peptides, diterpines, fluoroquinolones, hydrazines, ionophores, lincosamides, macrolides, organoarsenics, nitroimidazoles, penicillins, streptogramins, and sulfonamides in combination with various feeds used for those production species.

The present invention also includes cross-species metagenomic, proteomic, transcriptomic, and metabolomic analyses of the various GI microbiomes (e.g., stomach, rumen, small intestines, colon) for each livestock production species, including, but not limited to cattle, poultry (including raised game birds), swine, fish (including farmed fish), venison, bison, sheep, and goats, both before and after administration of combinations of antibiotics and antiprotozoal agents, including, but not limited to, those in the class of coccidiostats, aminoglycosides, cephalosporins, cyclic peptides, diterpines, fluoroquinolones, hydrazines, ionophores, lincosamides, macrolides, organoarsenics, nitroimidazoles, penicillins, streptogramins, and sulfonamides in combination with various feeds used for those production species.

The present invention includes the keystone species in enterotypes, and products derived from their administration or administration of their metabolic products, determined by cross-species comparison of the metagenomic, proteomic, transcriptomic, and metabolomic analyses of the various GI microbiomes (e.g., stomach, rumen, small intestines, colon), analysed in sequence of the passage of food in the digestive tract (first stomach or rumen, then small intestines, then colon) of the different livestock production species, including, but not limited to cattle, poultry (including raised game birds), swine, fish (including farmed fish), venison, bison, sheep, and goats, both before and after administration of an antibiotic, including, but not limited to, those in the class of aminoglycosides, cephalosporins, cyclic peptides, diterpines, fluoroquinolones, hydrazines, ionophores, lincosamides, macrolides, organoarsenics, nitroimidazoles, penicillins, streptogramins, and sulfonamides in combination with various feeds used for those production species, to determine a metabolic code for digestion through the solving of simultaneous equations with variables being bacterial strains with genes encoding metabolic products for food breakdown.

The present invention encompasses combinations of enterotype probiotic products or artificially optimized metabolic solutions (Bolsols) that are non-naturally occurring and involving inventive steps as contemplated in the intellectual property scheme described in Exhibit 1: Novel IP Regime: Patenting Microbial Ecologies and Exhibit 2: Metabols and Bolsols to Process Food.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings, such as attached FIG. 1 Syntheses), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1-2. (canceled)

3. A method for determining keystone bacterial species in the gut of a livestock animal for cross-species administration to a human subject comprising the steps of:
characterizing one or more natural bacterial enterotypes of a livestock;
administering one or more antibiotics to the livestock;
identifying one or more antibiotic administration-induced bacterial enterotypes;
comparing the antibiotic administration-induced bacterial enterotypes to the natural bacterial enterotypes to identify one or more keystone bacterial species; and
administering the one or more keystone bacterial species, and/or prebiotic metabolic products produced by the one or more keystone bacterial species, to a human subject.

4. The method of claim 3 wherein each of the bacterial enterotypes are representative of the gastrointestinal microbiomes in one or more of the stomach, rumen, small intestines, or colon of the livestock, and wherein the one or more keystone bacterial species are associated with a phenotypic effect on the livestock, and wherein the phenotypic effect is deposition of lean muscle over fat.

5. The method of claim 3 wherein the one or more antibiotics are selected from the group consisting of aminoglycosides, cephalosporins, cyclic peptides, diterpines, fluoroquinolones, hydrazines, ionophores, lincosam ides, macrolides, organoarsenics, nitroimidazoles, penicillins, streptogram ins, and sulfonamides.

6. The method of claim 3, wherein the one or more keystone bacterial species are formulated as a probiotic composition for administration to the human subject.

7. The method of claim 6, wherein the probiotic is co-administered to the human subject with food.

8. The method of claim 3, wherein the livestock is selected from the group consisting of cattle, poultry, swine, fish, sheep, goat, venison, and bison.

9. The method of claim 3, wherein each enterotype is characterized by one or more of metagenomic, proteomic, transcriptomic, and metabolomic analyses.

10. The method of claim 3, wherein the human subject is overweight, suffers from obesity, or is at risk for weight gain and/or obesity.

11. The method of claim 10, wherein the administration of the one or more keystone bacterial species effects the deposition of lean muscle over fat in the human subject to mitigate obesity and fat related weight gain.

12. The method of claim 3, wherein the at least one keystone bacterial species is used as a biological factory in a bioreactor to produce the prebiotic metabolic products.

13. The method of claim 16, wherein the administration of the prebiotic metabolic products to the human subject effects in the deposition of lean muscle over fat in the human subject to mitigate obesity and fat related weight gain.

14. A cross-species screening method for recapitulating a target bacterial enterotype in the gut of a human subject comprising the steps of:
characterizing a natural bacterial enterotype of a livestock;
administering one or more antibiotics to the livestock to yield a desired phenotype;
identifying an antibiotic administration-induced target bacterial enterotype associated with the desired phenotype;
screening for one or more compounds and/or drugs that recapitulate the target bacterial enterotype; and
administering the one or more compounds and/or drugs to a human subject as a prebiotic to recapitulate the target bacterial enterotype in the human subject.

15. The method of claim 14, wherein the one or more antibiotics are selected from the group consisting of aminoglycosides, cephalosporins, cyclic peptides, diterpines, fluoroquinolones, hydrazines, ionophores, lincosam ides, macrolides, organoarsenics, nitroimidazoles, penicillins, streptogram ins, and sulfonamides.

16. The method of claim 14 wherein the bacterial enterotype is representative of the gastrointestinal microbiome in one or more of the stomach, rumen, small intestines, or colon of the livestock.

17. The method of claim 14 wherein the desired phenotype is deposition of lean muscle over fat in the livestock.

18. The method of claim 14, wherein the livestock is selected from the group consisting of cattle, poultry, swine, fish, sheep, goat, venison, and bison.

19. The method of claim 14, wherein the administration of the prebiotic effects the deposition of lean muscle over fat in the human subject to mitigate obesity and fat related weight gain.

20. A cross-species metabolic composition for the deposition of lean muscle over fat in a human subject to mitigate obesity and fat related weight gain comprising one or more of a keystone bacterial species, a probiotic containing one or more keystone bacterial species, metabolic products from the one or more identified keystone bacterial species, or combinations thereof, each derived from a livestock animal.

21. The composition of claim 20, further comprising one or more components selected from the group consisting of vitamins, proteins, fatty acids, sugars, and compounds screened to modulate microbiota in various gastrointestinal microbiomes.

22. The composition of claim 20, wherein the keystone bacterial species, a probiotic containing one or more keystone bacterial species, metabolic products from the one or more identified keystone bacterial species, or combinations thereof, each derived from a livestock animal, are associated with an enterotype in the livestock animal that results in the deposition of lean muscle over fat.
